(19) United States
(12) Patent Application Publication
(10) Pub. No.: US 2026/0284181 A1
(43) Pub. Date: Sep. 24, 2026

(54) A viral vector and a vaccine composition comprising the vector

(71) Applicant: Rokote Laboratories Finland Oy, Kuopio (FI)

(72) Inventors: Kalle Saksela, Kuopio (FI); Seppo Ylä-Herttuala, Kuopio (FI); Kari Alitalo, Kuopio (FI); Riku Fagerlund, Kuopio (FI); Erkko Ylösmäki, Kuopio (FI)

(21) Appl. No.: 19/479,370

(22) PCT Filed: Apr. 29, 2024
(86) PCT No.: PCT/FI2024/050203

§ 371 Date: Oct. 28, 2025

(30) Foreign Application Priority Data
Apr. 28, 2023 (FI) 20235480

(51) Int. Cl. A61K 39/215; A61K 39/00; A61P 37/04; C07K 14/005; C12N 15/86

(52) U.S. Cl. CPC A61K 39/215; A61P 37/04; C07K 14/005; C12N 15/86; A61K 2039/5256; A61K 2039/543; C12N 2710/10343; C12N 2770/20022; C12N 2770/20034

(57) ABSTRACT

The present invention provides a viral vector comprising, at an insertion site in the viral genome, a nucleic acid sequence encoding a modified extracellular domain of the SARS-CoV-2 spike protein, wherein said modified extracellular domain comprises an N-terminal signal peptide causing the spike protein to enter a secretory system in a host cell, wherein said modified extracellular domain comprises a C-terminal deletion of at least of heptad repeat 2 (HR2), transmembrane segment (TM) and cytoplasmic tail (CT) of the spike protein. The present invention also provides a vaccination composition comprising said viral vector.

### FIELD

The present invention relates to coronavirus vaccines comprising a viral vector. Particularly, the present invention provides a vaccine against SARS-CoV-2.

### BACKGROUND

SARS-CoV-2, which emerged in December 2019, is responsible for the COVID-19 pandemic with devastating effects on health and economy worldwide. As of Feb. 28, 2023, a total of 758.3 million persons have been confirmed to be infected, and at least 6.8 million have been reported dead from the disease. Effective vaccines against new variants are urgently needed to reduce the global burden of mortality and morbidity associated with SARS-CoV-2 infection, and to enable gradual recovery from the lockdown of the societies.

More than 200 candidate vaccines are currently being developed, and promising and convincing data have been obtained from inactivated virus vaccines and protein subunit vaccines as well as RNA- and adenoviral vector-based vaccines expressing the SARS-CoV-2 spike protein. Currently, 12 COVID-19 vaccines have been granted a marketing authorization in the European union (EU), six mRNA-based vaccines, two adenovirus vector-based vaccines, one inactivated, adjuvanted vaccine, and three subunit vaccines. All these vaccines are administered via intramuscular route inducing mainly systemic antibody responses with limited induction of mucosal immunity in the airway, a prerequisite for sterilizing immunity. A booster vaccine designed to address this limitation is urgently needed.

### SUMMARY OF THE INVENTION

The invention is defined by the features of the independent claims. Some specific embodiments are defined in the dependent claims.

According to a first aspect of the present invention, there is provided a viral vector comprising, at an insertion site in the viral genome, a nucleic acid sequence encoding a modified extracellular domain of the SARS-CoV-2 spike protein,
wherein said modified extracellular domain comprises an N-terminal signal peptide causing the spike protein to enter a secretory system in a host cell,
wherein said modified extracellular domain comprises a C-terminal deletion of at least of heptad repeat 2 (HR2), transmembrane segment (TM) and cytoplasmic tail (CT) of the spike protein.

According to a second aspect of the present invention, there is provided a vaccine composition comprising the vector as defined in the present disclosure, preferably in a mucosal administration formulation.

According to a third aspect of the present invention, there is provided a method of preventing COVID-19 comprising administering a prophylactically or therapeutically effective amount of the vaccine composition as defined in the present disclosure to a mammal, preferably to a human.

According to a fourth aspect of the present invention, there is provided a use of a viral vector as defined in the present disclosure in the manufacturing of a vaccine composition for inducing an immune response in a mammal, preferably a human.

### BRIEF DESCRIPTION OF THE DRAWINGS

FIG. 1. Schematic presentation of the adenovirus-vectored vaccine construct comprising, at an E1 insertion site, a nucleic acid sequence encoding a modified extracellular domain of the SARS-CoV-2 spike protein with an IL-3 signal sequence and SV40 polyA signal under a human cytomegalovirus (CMV) promoter.

FIG. 2. Schematic presentation of the SARS-CoV-2 spike protein and modified extracellular domain (ectodomain). (A) A full-length SARS-CoV-2 spike protein corresponding to SEQ ID NO:1. Amino acid positions 1-13 correspond to a signal sequence (SS), amino acids 14-1205 correspond to the extracellular domain (ectodomain) of the spike protein, amino acids 1209-1229 correspond to the transmembrane domain (TM) of the spike protein and amino acids 1230-1268 correspond to cytoplasmic tail (CT) of the spike protein. NTD, N-terminal domain; RBD, receptor binding domain, SD1, subdomain; SD2, subdomain 2; S1/S2, S1/S2 cleavage site; S2′, S2′ cleavage site; FP, fusion peptide; HR1 heptad repeat 1; CH, central helix region; CD, connector domain; HR2; heptad repeat 2. (B) The extracellular domain (ectodomain) of the spike protein without TM and CT regions (amino acids 1-1203). (C) The extracellular domain (ectodomain) of the spike protein without HR2, TM and CT regions.

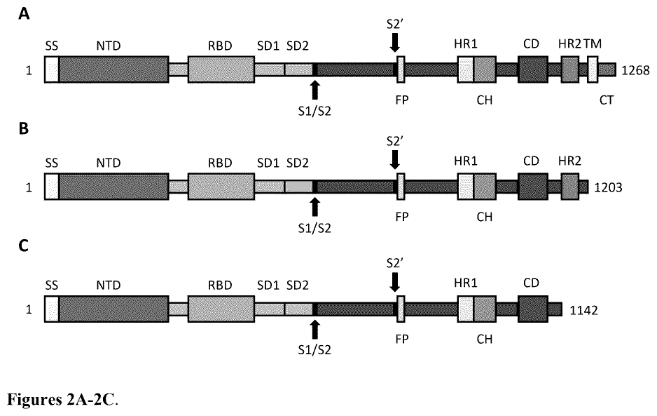

FIG. 3 shows western blot analysis of supernatant collected from pFCV2.1 transfected HEK293 cells. pGFP was similarly transfected and used to control antibody specificity. 25 nanograms of purified spike protein was used as a positive control.

FIG. 4 shows vaccination and blood sampling schedule (A) in Syrian golden hamsters and immunological responses at 14 days postinfection (B) and 28 days postinfection (C). Hamsters were intranasally (i.n.) vaccinated with four vaccine doses ranging from 2.5×10⁵ to 2.5×10⁸ virus particles/dose, and the immunological IgG response against the original strain (Wuhan) receptor binding domain (RBD) and the omicron BA.4/5 RBD were measured by ELISA assay.

FIG. 5. Schematic presentation of the modified ectodomain of SARS-CoV-2 spike protein with different fusion domains linked to the C-terminus. (A) The modified ectodomain of the spike protein with C-terminally fused trimerization domain. (B) The modified ectodomain of the spike protein with C-terminally fused transmembrane domain. (C) The modified ectodomain of the spike protein with C-terminally fused trimerization and transmembrane domains. (D) The modified ectodomain of the spike protein with one or more receptor binding domains (RBD) from other, immunologically distinct, SARS-CoV-2 spike proteins fused to the C-terminus. (E) The modified ectodomain of the spike protein with one or more receptor binding domains (RBD) from other, immunologically distinct, SARS-CoV-2 spike proteins and trimerization domain fused to the C-terminus. (F) The modified ectodomain of the spike protein with one or more receptor binding domains (RBD) from other, immunologically distinct, SARS-CoV-2 spike proteins and trimerization domain fused to the C-terminus of the modified ectodomain. (G) The modified ectodomain of the spike protein with one or more receptor binding domains (RBD) from other, immunologically distinct, SARS-CoV-2 spike proteins and transmembrane domain fused to the C-terminus. (H) The modified ectodomain of the spike protein with one or more receptor binding domains (RBD) from other, immunologically distinct, SARS-CoV-2 spike proteins with trimerization and transmembrane domains fused to the C-terminus. TRD, trimerization domain, TMD, transmembrane domain, RBD, receptor binding domain.

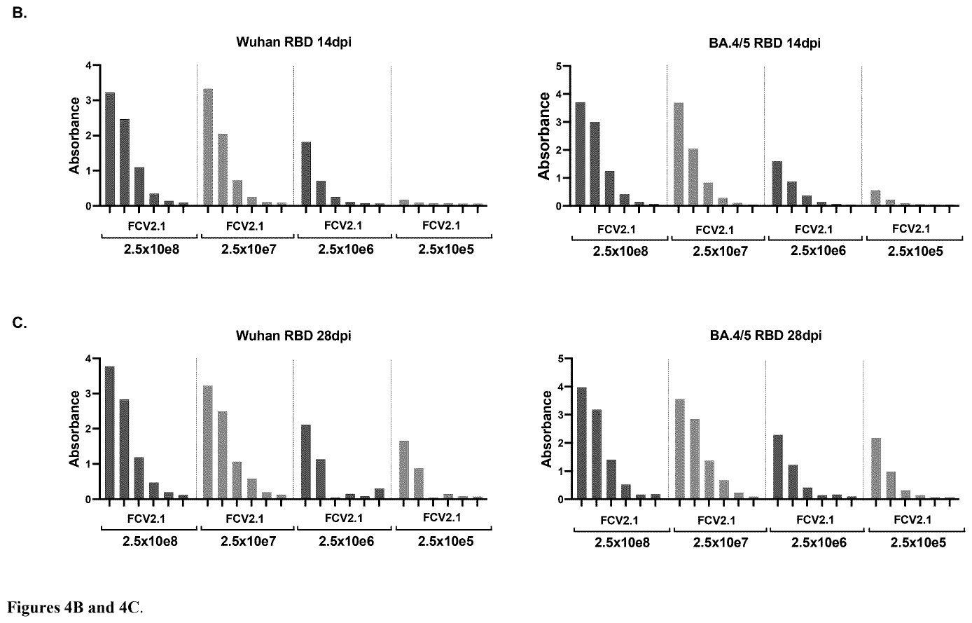

FIG. 6. Intranasal vaccination with FCV2.1 protects hamsters from SARS-CoV-2 infection. A.) Immunization and challenge schedule. Viral loads B.), infectious titer C.), and immunohistochemical analysis of viral nucleocapsid D.) were assessed from nasal turbinates in FCV2.1-vaccinated (2 doses, 1×10¹⁰ VP/dose) and non-vaccinated hamsters. In FCV2.1-vaccinated hamsters, the level of SARS-CoV-2 virus decreased very rapidly, approaching a detection limit. This indicates the potential for FCV2.1, in addition to protecting vaccinated individuals, to significantly reduce transmission and virus spread.

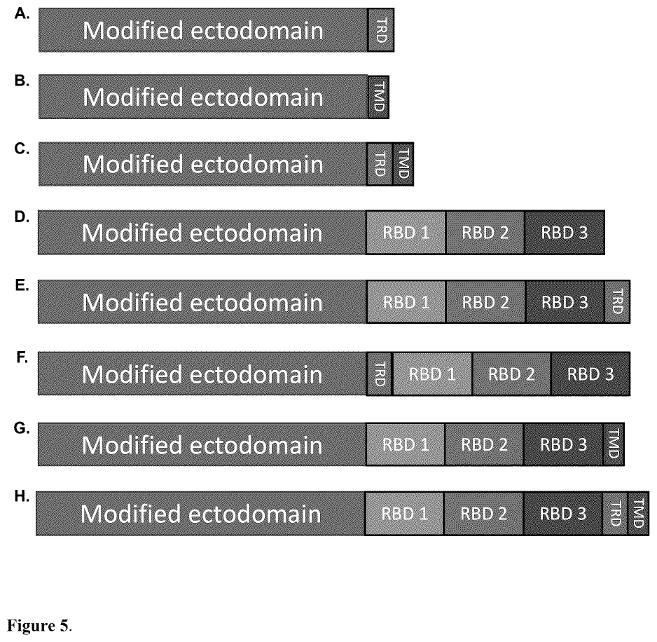

FIG. 7. Schematic presentation of FCV2.3 and neutralization efficacy when administered as a booster dose after primary dose of Comirnaty (Original) A.) FCV2.3 consists of the modified ectodomain of the spike protein with one receptor binding domain (RBD1) from other, immunologically distinct, SARS-CoV-2 spike protein fused to the C-terminus. The receptor binding domain in FCV2.3 is from the XBB variant. B.) Neutralizing antibody (NAb) response of mice receiving two intramuscular doses of Comirnaty (Com (w)) and mice receiving one intramuscular dose of Comirnaty followed by one intranasal dose of FCV2.3 (FCV2.3). As opposed to two doses of Comirnaty, one booster dose of FCV2.3 induces robust NAb responses against all tested variants (Wuhan, BA.4/5 and XBB). Administration of FCV2.3 as a booster after primary vaccination with Comirnaty results in robust neutralizing antibody responses against multiple SARS-CoV-2 variants, including the XBB variant, indicating that the addition of an RBD from an immunologically distinct variant can broaden the NAb response to allow protection against a broader range of SARS-CoV-2 variants.

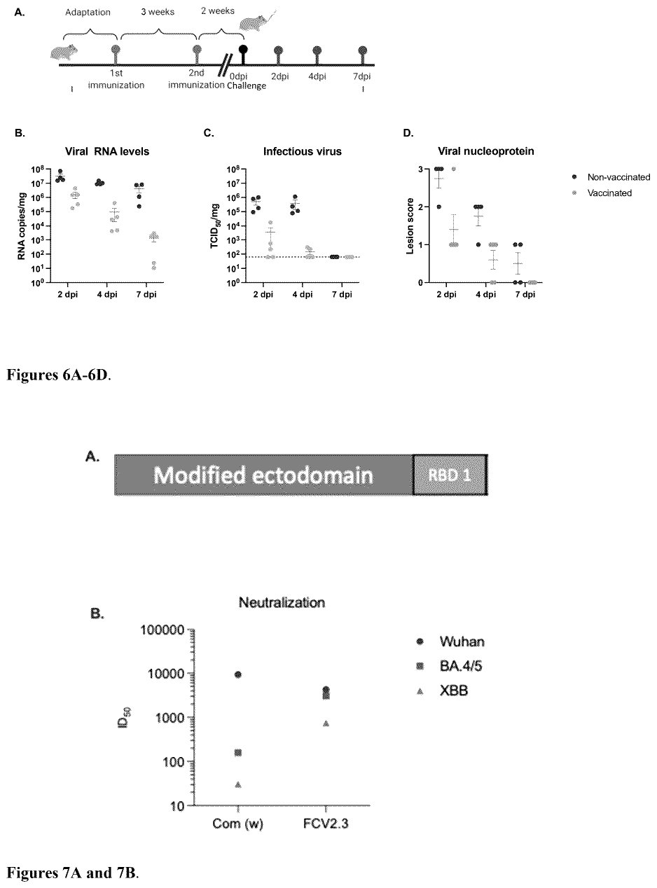

### EMBODIMENTS

In the present context, the term “vector” is used to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Useful viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid. Thus, the term viral vector may refer either to a virus or viral particle capable of transferring a nucleic acid into a cell or to the transferred nucleic acid itself.

The term “signal sequence” means herein a signal peptide that is a part of the N-terminus of a secretory protein that is secreted outside a cell and thus passes through the cell membrane. The signal peptide is usually composed of approximately 10 to 30 amino acids, and is subsequently cleaved and removed by a protease specific for the cell membrane, and only the secretory protein is transferred outside the cell. Signal peptides serve as targeting signals, enabling cellular transport machinery to direct proteins to specific intracellular or extracellular locations. To date, more than 4000 signal peptides present in eukaryotic cells are known. DNA libraries encoding signal peptides are disclosed, e.g., in WO2021045541A1 and KR20210028116A.

The term “transmembrane domain” refers herein to a hydrophobic alpha helix structure that transverses the host cell membrane. The transmembrane domain may be directly fused to the C-terminal part of the fusion protein encoded by a vector. For example, the transmembrane domain may be derived from an integral membrane protein (e.g., receptor, cluster of differentiation molecule, enzyme, transporter, cell adhesion molecule, or the like). A particular example is the transmembrane domain derived from Type 1 transmembrane proteins such as human VCAM-1 protein (vascular cell adhesion molecule 1). Type I transmembrane proteins are anchored to the lipid membrane with a stop-transfer anchor sequence and have their N-terminal domains targeted to the extracellular space, when a mature form of the protein is located on the cell membrane.

The present invention is based on a viral vector producing a modified version of the SARS-CoV-2 spike protein, which has an improved yield when expressed from a viral vector, preferably from an adenoviral vector. Improved expression of the spike protein provides stronger immunogenicity for the vaccine. Further, the C-terminal deletion of the modified spike protein removes an immunodominant epitope potentially competing with neutralizing epitopes of the RBD region for immune response thus the present modified spike protein directs anti-spike immune responses more to the remaining spike ectodomain containing the neutralizing epitopes of the RBD region.

Viral vectors represent widely used platform for vaccine design, and they have shown great potential in the fight against a variety of infectious diseases. Currently there are numerous viral vector-based vaccines against SARS-CoV-2 in pre-clinical and clinical development including two vaccines with market approval in the EU.

The vaccine of the present invention has been developed to suite as a booster vaccine for those who have received full vaccination cycle (1 or more doses) of for example Comirnaty, Spikevax, or ADZ1222 as well as a primary vaccine for those who have not yet received any vaccine against SARS-CoV-2 infection. The present viral vector in said vaccine produces a secreted version of a C-terminally truncated SARS-CoV-2 spike protein. In a preferred embodiment, the C-terminally truncated spike protein comprises mutated furin cleavage site with single-amino acid modifications R677G, R678S and R680S (corresponding to positions 685, 686 and 688 in SEQ ID NO:2, respectively). In an embodiment, the C-terminally truncated spike protein has been modified by proline substitutions at residues 981 and 982 (corresponding to positions 989 and 990 in SEQ ID NO:2, respectively).

The spike protein mediates the entry of SARS-CoV-2 into the host cell and is the main target of neutralizing antibodies arising in infected individuals, and a predominant target of antibody-mediated immunity (Chaudhary et al., 2021). Therefore, apart from inactivated and attenuated whole virus approaches, virtually all ongoing COVID-19 vaccine projects use the spike protein as the immunogen. All COVID-19 vaccines authorized in EU and in the United States are based on the spike protein of SARS-CoV-2.

The omicron BA.5 strain spike protein has the following amino acid sequence (SEQ ID NO:1):

| | |
|---|---|
| MFVFLVLLPLVSSQCVNLITRTQSYTNSFTRGVYYPDKVFRSSVLHSTQDLFLPFFSNVT | 60 |
| WFHAISGINGIKRFDNPVLPFNDGVYFASTEKSNIIRGWIFGTTLDSKTQSLLIVNNATN | 120 |
| VVIKVCEFQFCNDPFLDVYYHKNNKSWMESEFRVYSSANNCTFEYVSQPFLMDLEGKQGN | 180 |
| FKNLREFVFKNIDGYFKIYSKHTPINLGRDLPQGFSALEPLVDLPIGINITRFQTLLALH | 240 |
| RSYLTPGDSSSGWTAGAAAYYVGYLQPRTFLLKYNENGTITDAVDCALDPLSETKCTLKS | 300 |
| FTVEKGIYQTSNFRVQPTESIVRFPNITNLCPFDEVFNATRFASVYAWNRKRISNCVADY | 360 |
| SVLYNFAPFFAFKCYGVSPTKLNDLCFTNVYADSFVIRGNEVSQIAPGQTGNIADYNYKL | 420 |
| PDDFTGCVIAWNSNKLDSKVGGNYNYRYRLFRKSNLKPFERDISTEIYQAGNKPCNGVAG | 480 |
| VNCYFPLQSYGFRPTYGVGHQPYRVVVLSFELLHAPATVCGPKKSTNLVKNKCVNFNENG | 540 |
| LTGTGVLTESNKKFLPFQQFGRDIADTTDAVRDPQTLEILDITPCSFGGVSVITPGTNTS | 600 |
| NQVAVLYQGVNCTEVPVAIHADQLTPTWRVYSTGSNVFQTRAGCLIGAEYVNNSYECDIP | 660 |
| IGAGICASYQTQTKSHRRARSVASQSIIAYTMSLGAENSVAYSNNSIAIPTNFTISVTTE | 720 |
| ILPVSMTKTSVDCTMYICGDSTECSNLLLQYGSFCTQLKRALTGIAVEQDKNTQEVFAQV | 780 |
| KQIYKTPPIKYFGGFNFSQILPDPSKPSKRSFIEDLLFNKVTLADAGFIKQYGDCLGDIA | 840 |
| ARDLICAQKFNGLTVLPPLLTDEMIAQYTSALLAGTITSGWTFGAGAALQIPFAMQMAYR | 900 |
| FNGIGVTQNVLYENQKLIANQFNSAIGKIQDSLSSTASALGKLQDVVNHNAQALNTLVKQ | 960 |
| LSSKFGAISSVLNDILSRLDKVEAEVQIDRLITGRLQSLQTYVTQQLIRAAEIRASANLA | 1020 |
| ATKMSECVLGQSKRVDFCGKGYHLMSFPQSAPHGVVFLHVTYVPAQEKNFTTAPAICHDG | 1080 |
| KAHFPREGVFVSNGTHWFVTQRNFYEPQIITTDNTFVSGNCDVVIGIVNNTVYDPLQPEL | 1140 |
| DSFKEELDKYFKNHTSPDVDLGDISGINASVVNIQKEIDRLNEVAKNLNESLIDLQELGK | 1200 |
| YEQYIKWPWYIWLGFIAGLIAIVMVTIMLCCMTSCCSCLKGCCSCGSCCKFDEDDSEPVL | 1260 |
| KGVKLHYT | 1268 |

In the above sequence (SEQ ID NO:1), amino acid positions 1-13 correspond to a signal sequence, amino acids 14-1203 correspond to the extracellular domain (ectodomain) of the spike protein, amino acids 1209-1229 correspond to the transmembrane domain of the spike protein and amino acids 1230-1268 correspond to cytoplasmic region of the spike protein.

In a preferred embodiment, the modified extracellular domain of the SARS-CoV-2 spike protein has the following amino acid sequence (SEQ ID NO:2):

| | |
|---|---|
| MSRLPVLLLLQLLVRPGLQAPQCVNLITRTQSYTNSFTRGVYYPDKVFRSSVLHSTQDLF | 60 |
| LPFFSNVTWFHAISGTNGTKRFDNPVLPFNDGVYFASTEKSNIIRGWIFGTTLDSKTQSL | 120 |
| LIVNNATNVVIKVCEFQFCNDPFLDVYYHKNNKSWMESEFRVYSSANNCTFEYVSQPFLM | 180 |
| DLEGKQGNFKNLREFVFKNIDGYFKIYSKHTPINLGRDLPQGFSALEPLVDLPIGINITR | 240 |
| FQTLLALHRSYLTPGDSSSGWTAGAAAYYVGYLQPRTFLLKYNENGTITDAVDCALDPLS | 300 |
| ETKCTLKSFTVEKGIYQTSNFRVQPTESIVRFPNITNLCPFDEVFNATRFASVYAWNRKR | 360 |
| ISNCVADYSVLYNFAPFFAFKCYGVSPTKLNDLCFTNVYADSFVIRGNEVSQIAPGQTGN | 420 |
| IADYNYKLPDDFTGCVIAWNSNKLDSKVGGNYNYRYRLFRKSNLKPFERDISTEIYQAGN | 480 |
| KPCNGVAGVNCYFPLQSYGFRPTYGVGHQPYRVVVLSFELLHAPATVCGPKKSTNLVKNK | 540 |
| CVNFNFNGLTGTGVLTESNKKFLPFQQFGRDIADTTDAVRDPQTLEILDITPCSFGGVSV | 600 |
| ITPGTNTSNQVAVLYQGVNCTEVPVAIHADQLTPTWRVYSTGSNVFQTRAGCLIGAEYVN | 660 |
| NSYECDIPIGAGICASYQTQTKSHGSASSVASQSIIAYTMSLGAENSVAYSNNSIAIPTN | 720 |
| FTISVTTEILPVSMTKTSVDCTMYICGDSTECSNLLLQYGSFCTQLKRALTGIAVEQDKN | 780 |
| TQEVFAQVKQIYKTPPIKYFGGFNFSQILPDPSKPSKRSFIEDLLENKVTLADAGFIKQY | 840 |
| GDCLGDIAARDLICAQKFNGLTVLPPLLTDEMIAQYTSALLAGTITSGWTFGAGAALQIP | 900 |
| FAMQMAYRFNGIGVTQNVLYENQKLIANQFNSAIGKIQDSLSSTASALGKLQDVVNHNAQ | 960 |
| ALNTLVKQLSSKFGAISSVLNDILSRLDPPEAEVQIDRLITGRLQSLQTYVTQQLIRAAE | 1020 |
| IRASANLAATKMSECVLGQSKRVDFCGKGYHLMSFPQSAPHGVVFLHVTYVPAQEKNFTT | 1080 |
| APAICHDGKAHFPREGVFVSNGTHWFVTQRNFYEPQIITTDNTFVSGNCDVVIGIVNNTV | 1140 |
| YD | 1142 |

In the above sequence (SEQ ID NO:2), amino acid positions 1-19 correspond to a signal sequence, and amino acids 22-1142 correspond to the extracellular domain (ectodomain) of the spike protein with a deletion at the C-terminal end. In another embodiment, the C-terminal deletion site is between the connector domain, CD, (amino acids 1030-1062 of SEQ ID NO:1) and the heptad repeat 2, HR2 (amino acids 1158-1197 of SEQ ID NO:1). In another embodiment, the C-terminal deletion site is in the connector domain, CD, (amino acids 1030-1062 of SEQ ID NO:1). In another preferred embodiment, the deletion site of said C-terminal deletion is located at the region corresponding to positions 1030-1157 of the omicron BA.5 strain spike protein of SEQ ID NO:1.

Accordingly, the present invention is directed to a viral vector comprising, at an insertion site in the viral genome, a nucleic acid sequence encoding a modified extracellular domain of the SARS-CoV-2 spike protein,
wherein said modified extracellular domain comprises an N-terminal signal peptide causing the spike protein to enter a secretory system in a host cell,
wherein said modified extracellular domain comprises a C-terminal deletion of at least of heptad repeat 2 (HR2), transmembrane segment (TM) and cytoplasmic tail (CT) of the spike protein.

In a preferred embodiment, said modified extracellular domain of the SARS-CoV-2 spike protein corresponding to a C-terminally deleted spike ectodomain comprises the amino acid sequence of SEQ ID NO:2, amino acids 22-1142 of SEQ ID NO:2 or a sequence which has at least 80% sequence identity with the sequence of SEQ ID NO:2 or amino acids 22-1142 of SEQ ID NO:2. In a more preferred embodiment, said modified extracellular domain of the SARS-CoV-2 spike protein corresponding to a C-terminally deleted spike ectodomain comprises a sequence which has at least 85%, 90%, 95%, 96%, 97%, 98%, 99% or 99.5% sequence identity with the sequence of SEQ ID NO:2 or amino acids 22-1142 of SEQ ID NO: 2.

The term “sequence identity,” in the context of two or more amino acid sequences, refers to two or more sequences or subsequences that are the same. Two sequences are “substantially identical” if two sequences have a specified percentage of amino acid residues that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection.

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. For amino acid sequences, the BLASTP program uses as defaults a wordlength of 3, and expectation (E) of 10, and the BLOSUM62 scoring matrix [see Henikoff and Henikoff, (1992) Proc Natl Acad Sci USA 89(22):10915-10919] alignments (B) of 50, expectation (E) of 10, M=5, N=−4, and a comparison of both strands. For short amino acid sequences, PAM30 scoring matrix can be applied.

In another preferred embodiment, said N-terminal signal peptide is the human interleukin-3 secretion signal peptide or the natural signal peptide of the SARS-CoV-2 spike protein (i.e. amino acids 1-13 of SEQ ID NO:1).

In another preferred embodiment, said modified extracellular domain of the SARS-CoV-2 spike protein comprises a mutated furin cleavage site with single amino acid modifications in positions R677G, R678S and R680S (corresponding to positions 685, 686 and 688 in SEQ ID NO:2, respectively), and/or proline substitutions at residues 981 and 982 (corresponding to positions 989 and 990 in SEQ ID NO:2, respectively).

In another preferred embodiment, said modified extracellular domain of the SARS-CoV-2 spike protein comprises the amino acid sequence of SEQ ID NO:2 or amino acids 22-1142 of SEQ ID NO:2.

In another preferred embodiment, said vector is selected from the group consisting of adenovirus vectors, adeno-associated virus vectors, retrovirusvectors, lentivirus vectors, herpex simplex virus vectors, poxvirus vectors, preferably vaccinia virus vectors and orthomyxovirus vectors, preferably influenza virus vectors.

In a more preferred embodiment, said vector is an adenoviral vector, most preferably a non-replicating adenovirus serotype 5 vector. In some embodiments, an adenoviral vector as described herein is adenovirus 5 (Ad5), which can include, for example, Ad5 with deletions of the E1/E3 regions and Ad5 with a deletion of the E4 region. Other suitable adenoviral vectors include strains 2, orally tested strains 4 and 7, enteric adenoviruses 40 and 41, and other human adenovirus strains (e.g. Ad34) as well as chimpanzee adenovirus 36 and serotype Y25 and vectors derived from a group C gorilla adenovirus that are sufficient for delivering an antigen and eliciting an adaptive immune response to the transgene antigen. In an embodiment, the insertion site in said adenovirus serotype 5 vector is selected from the group consisting of an E1 insertion site, an E3 insertion site, and an E4 insertion site.

Various promoters can be used in the adenoviral vectors described herein. For example, commonly used promoters and enhancers are derived, e.g., from beta-actin, adenovirus, simian virus (SV40), and human cytomegalovirus (CMV). For example, vectors allowing expression of proteins under the direction of the CMV promoter, beta-actin promoter, SV40 early promoter, SV40 later promoter, metallothionein promoter, murine mammary tumor virus promoter,

In another preferred embodiment, said nucleic acid sequence encoding said modified extracellular domain is as set forth in SEQ ID NO:3, or a nucleotide sequence having at least 70%, 80%, 85%, 90%, or 95% sequence identity with the nucleotide sequence as set forth in SEQ ID NO:3.

In a preferred embodiment, the modified extracellular domain of the SARS-CoV-2 spike protein is encoded by the following nucleotide sequence (SEQ ID NO:3):

| | |
|---|---|
| ATGAGCCGCCTGCCCGTCCTGCTCCTGCTCCAACTCCTGGTCCGCCCCGGACTCCAAGCT | 60 |
| CCCCAGTGCGTCAACCTGATCACCAGAACCCAAAGCTACACCAATAGCTTCACACGGGGG | 120 |
| GTGTACTACCCTGATAAGGTGTTCCGGAGTTCTGTGCTGCACTCCACACAGGACCTGTTT | 180 |
| CTGCCTTTTTTTTCCAATGTGACATGGTTCCACGCCATCAGCGGCACCAACGGCACAAAA | 240 |
| AGGTTTGACAACCCCGTGCTGCCATTTAATGATGGGGTGTACTTCGCTTCCACCGAGAAA | 300 |
| TCCAACATCATTAGGGGCTGGATCTTCGGCACTACCCTGGATTCTAAGACTCAGTCCCTG | 360 |
| CTGATCGTGAATAACGCCACCAACGTGGTGATCAAGGTGTGCGAATTTCAGTTCTGCAAC | 420 |
| GATCCATTCCTGGACGTGTACTACCACAAGAATAACAAGAGCTGGATGGAGAGCGAGTTC | 480 |
| CGCGTGTACTCTTCCGCAAACAACTGCACCTTTGAATATGTGAGCCAGCCCTTCCTGATG | 540 |
| GACCTGGAGGGAAAGCAGGGCAATTTCAAAAACCTCAGGGAATTTGTCTTCAAGAATATC | 600 |
| GATGGCTATTTCAAGATCTACAGCAAGCATACTCCCATCAACCTGGGCAGAGACCTGCCC | 660 |
| CAGGGCTTCAGCGCTCTGGAGCCTCTGGTGGACCTGCCCATCGGCATCAACATTACCAGA | 720 |
| TTCCAGACCCTGCTGGCTCTGCATCGGTCTTACCTGACTCCCGGAGATAGCAGCAGCGGC | 780 |
| TGGACCGCCGGCGCCGCTGCCTATTATGTGGGGTACCTGCAGCCTAGAACCTTCCTGCTG | 840 |
| AAGTACAACGAGAACGGCACCATCACAGATGCCGTGGACTGCGCTCTGGACCCACTCTCC | 900 |
| GAGACCAAATGCACCCTGAAGTCCTTTACCGTGGAGAAAGGCATCTACCAGACCTCCAAT | 960 |
| TTCAGAGTGCAGCCTACAGAGAGCATCGTGCGCTTCCCCAACATCACCAACCTGTGCCCT | 1020 |
| TTCGACGAGGTGTTCAATGCAACACGGTTTGCCAGCGTGTACGCTTGGAATCGGAAGCGG | 1080 |
| ATCAGCAACTGCGTGGCCGACTACTCTGTGCTGTATAACTTCGCCCCCTTTTTCGCCTTT | 1140 |
| AAATGTTACGGAGTGTCCCCAACTAAGCTGAACGACCTGTGCTTCACAAACGTGTATGCT | 1200 |
| GATAGCTTCGTGATTCGGGGCAACGAAGTCAGCCAGATCGCTCCCGGACAGACTGGAAAC | 1260 |
| ATCGCCGACTACAACTACAAGCTGCCTGACGACTTCACCGGCTGCGTGATCGCCTGGAAC | 1320 |
| TCCAACAAGCTGGATAGCAAGGTGGGCGGCAATTACAACTACCGGTATCGCCTGTTCAGA | 1380 |
| AAGTCTAATCTGAAGCCTTTTGAGCGCGACATCAGCACCGAGATCTATCAGGCCGGAAAC | 1440 |
| AAGCCCTGCAACGGAGTGGCCGGGGTGAACTGCTACTTTCCTCTGCAGAGCTATGGCTTT | 1500 |
| CGGCCCACCTACGGGGTGGGGCACCAGCCCTACCGGGTGGTGGTCCTGTCCTTCGAGCTG | 1560 |
| CTGCACGCCCCCGCCACCGTGTGCGGCCCCAAGAAATCCACCAACCTGGTGAAGAATAAG | 1620 |
| TGTGTGAACTTCAACTTCAACGGCCTCACCGGCACCGGCGTGCTGACCGAGTCTAATAAG | 1680 |
| AAATTCCTGCCCTTCCAGCAGTTCGGGAGGGACATCGCCGATACCACAGATGCCGTCAGA | 1740 |
| GACCCACAGACCCTGGAGATCCTGGACATCACCCCCTGCTCATTCGGCGGCGTGAGCGTC | 1800 |
| ATCACACCTGGCACCAACACCAGCAATCAGGTTGCCGTGCTGTACCAGGGGGTGAACTGC | 1860 |
| ACCGAGGTGCCTGTGGCCATTCACGCCGACCAGCTCACCCCTACTTGGCGGGTGTACAGC | 1920 |
| ACAGGCTCCAACGTGTTCCAGACCAGGGCCGGATGCCTGATCGGCGCCGAGTACGTGAAT | 1980 |
| AATTCCTATGAGTGCGACATCCCCATCGGAGCCGGGATTTGCGCCAGCTACCAGACCCAG | 2040 |
| ACAAAGAGCCACGGCAGCGCCAGCTCCGTCGCCAGCCAGAGCATCATCGCCTACACCATG | 2100 |
| TCCCTGGGGGCCGAGAATAGCGTCGCATACAGTAATAATTCCATTGCCATCCCAACCAAC | 2160 |
| TTCACCATCAGCGTGACCACCGAAATTCTGCCCGTGTCAATGACCAAAACCTCCGTGGAC | 2220 |
| TGTACCATGTACATCTGTGGCGACAGCACCGAGTGCAGCAACCTGCTGCTGCAGTACGGA | 2280 |
| TCCTTTTGCACCCAGCTGAAGCGGGCCCTGACAGGAATCGCCGTGGAGCAGGACAAGAAC | 2340 |
| ACCCAGGAGGTGTTCGCCCAGGTGAAACAGATCTACAAGACCCCCCCCATCAAGTACTTC | 2400 |
| GGTGGGTTCAACTTCAGCCAGATCCTGCCAGACCCCTCTAAGCCCTCCAAGAGGAGCTTT | 2460 |
| ATCGAAGACCTGCTGTTCAACAAGGTCACCCTGGCCGATGCAGGCTTCATCAAGCAGTAC | 2520 |
| GGGGATTGTCTGGGCGACATCGCCGCCAGAGACCTGATCTGTGCACAGAAGTTTAACGGC | 2580 |
| CTGACAGTCCTGCCTCCACTGCTGACTGACGAGATGATCGCCCAGTATACCTCCGCCCTG | 2640 |
| CTGGCCGGAACTATTACTAGCGGCTGGACCTTCGGCGCCGGTGCCGCTCTGCAGATCCCC | 2700 |
| TTTGCCATGCAGATGGCCTATCGCTTCAACGGCATTGGAGTGACCCAGAACGTGCTGTAT | 2760 |
| GAGAACCAGAAGCTGATCGCCAACCAGTTCAACTCCGCCATCGGCAAAATTCAGGACTCT | 2820 |
| CTGAGCTCCACTGCCAGTGCCCTGGGCAAGCTGCAGGATGTGGTGAATCACAACGCCCAG | 2880 |
| GCCCTGAACACACTCGTGAAACAGCTGTCCTCCAAGTTCGGCGCTATTAGCAGCGTGCTG | 2940 |
| AATGATATCCTGAGCCGGCTGGATCCCCCCGAGGCCGAAGTGCAGATTGATAGACTGATT | 3000 |
| ACAGGCAGACTGCAGAGTCTGCAGACCTACGTGACTCAGCAGCTGATCCGCGCTGCCGAG | 3060 |
| ATCAGAGCCTCCGCCAACCTGGCCGCCACCAAGATGTCCGAGTGCGTGCTGGGCCAGTCC | 3120 |
| AAAAGAGTGGATTTTTGCGGCAAGGGCTACCACCTGATGTCATTTCCTCAGTCTGCTCCT | 3180 |
| CACGGCGTGGTGTTCCTGCACGTGACCTATGTGCCCGCCCAGGAGAAGAACTTTACCACT | 3240 |
| GCCCCTGCCATCTGCCACGACGGCAAGGCCCACTTTCCCCGCGAGGGCGTGTTCGTGTCT | 3300 |
| AACGGAACCCACTGGTTCGTCACCCAGAGGAATTTTTACGAGCCACAGATCATTACCACA | 3360 |
| GATAACACCTTTGTCAGTGGCAATTGCGATGTGGTGATCGGCATCGTCAACAATACCGTG | 3420 |
| TACGAC | 3426 |

In another preferred embodiment, said SARS-CoV-2 spike protein expressed by said vector is secreted as a soluble monomer from a host cell infected by the viral vector.

In another preferred embodiment, said modified extracellular domain of the SARS-CoV-2 spike protein comprises in the C-terminal end one or more further receptor binding domains (RBD) from SARS-CoV-2 spike proteins which are immunologically distinct from said modified extracellular domain of the SARS-CoV-2 spike protein. For example, in an embodiment said modified extracellular domain of the SARS-CoV-2 spike protein corresponds immunologically to the omicron BA.5 strain spike protein, wherein said one or more further receptor binding domain (RBD) from SARS-CoV-2 spike proteins correspond(s) immunologically to the spike protein(s) derived from the group of SARS-CoV-2 variants consisting of: XBB.1.5, XBB.1.16, XBB.1.9.1, XBB.1.9.2, XBB.1.5.1, FD.2, XBB, BQ.1.1, CH.1.1, BQ.1, BN.1 and BJ.1.

In another preferred embodiment, said modified extracellular domain of the SARS-CoV-2 spike protein comprises in the C-terminal end a trimerization domain, preferably T4 fibritin trimerization domain, human collagen trimerization domain, leuzine zipper domain or any other additional C-terminal amino acid sequence promoting trimerization of said spike protein.

In another preferred embodiment, said modified extracellular domain of the SARS-CoV-2 spike protein comprises in the C-terminal end a transmembrane domain, or any other additional C-terminal amino acid sequence promoting transmembrane anchoring of said spike protein.

In another more preferred embodiment, said modified extracellular domain of the SARS-CoV-2 spike protein comprises in the C-terminal region in any order i) a trimerization domain, preferably T4 fibritin trimerization domain, human collagen trimerization domain, leuzine zipper domain or any other additional C-terminal amino acid sequence promoting trimerization of said spike protein, and ii) a transmembrane domain, or any other additional C-terminal amino acid sequence promoting transmembrane anchoring of said spike protein.

In another more preferred embodiment, said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, and ii) a trimerization domain, preferably T4 fibritin trimerization domain, human collagen trimerization domain, leuzine zipper domain or any other additional C-terminal amino acid sequence promoting trimerization of said spike protein.

In another more preferred embodiment, said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, and ii) a transmembrane domain, or any other additional C-terminal amino acid sequence promoting transmembrane anchoring of said spike protein.

In another more preferred embodiment, said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, ii) a trimerization domain, preferably T4 fibritin trimerization domain, human collagen trimerization domain, leuzine zipper domain or any other additional C-terminal amino acid sequence promoting trimerization of said spike protein, and iii) a transmembrane domain or any other additional C-terminal amino acid sequence promoting transmembrane anchoring of said spike protein.

In an embodiment, the present invention is also directed to a vaccine composition comprising the viral vector as defined in the present disclosure, preferably in a mucosal administration formulation.

In a preferred embodiment, said mucosal administration formulation is selected from the group consisting of nasal drops, aerosols, sprays, powder sprays, gels, microspheres, liposomes, membranes, and suspensions. More preferably, said vaccine composition is a spray or sprayable composition in a liquid dosage form. In one embodiment, said vaccine composition is to be administered intranasally.

In a preferred embodiment, the vaccine further comprises a pharmaceutically-acceptable adjuvant, carrier, diluent or excipient. In one more preferred embodiment, said vaccine composition comprises A195 buffer, Tris, sodium chloride, magnesium chloride, histidine, sucrose, polysorbate-80, EDTA, and ethanol. In a most preferred embodiment, said vaccine composition comprises A195 buffer; 10 mM Tris at a pH of 7.4, 75 mM NaCl, 1 mM MgCl₂, 10 mM histidine, 5% (wt/vol) sucrose, 0.02% polysorbate-80 (wt/vol), 0.1 mM EDTA, and 0.5% (vol/vol) ethanol).

In a further embodiment, the present invention is directed to a method of preventing COVID-19 comprising administering a prophylactically or therapeutically effective amount of the vaccine composition as defined in the present disclosure to a mammal, preferably a human.

In a still further embodiment, the present invention is directed to a use of a viral vector as defined in the present disclosure in the manufacturing of a pharmaceutical composition for inducing an immune response in a mammal, wherein said vector encodes a modified extracellular domain of the SARS-CoV-2 spike protein,
wherein said modified extracellular domain comprises an N-terminal signal peptide causing the spike protein to enter a secretory system in a host cell,
wherein said modified extracellular domain comprises a C-terminal deletion of at least of heptad repeat 2 (HR2), transmembrane segment (TM) and cytoplasmic tail (CT) of the spike protein.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### Experimental Section

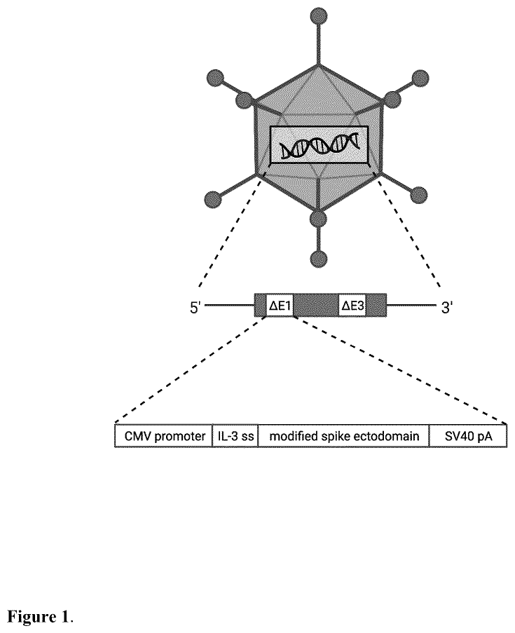

This Experimental Section provides results obtained with the investigational medicinal product (IMP) FINCoVac 2.1 (FCV2.1), which is based on a non-replicating Ad5 vector expressing a modified extracellular domain (ectodomain) of the SARS-CoV-2 spike protein from the omicron BA.5 strain (FIG. 1). FINCoVac 2.1 is designed for intranasal administration, and when administered into the nasal cavity, it was anticipated that the encoded modified spike ectodomain will generate a host immune reaction leading to immunity to SARS-CoV-2. Also, this section provides results obtained with a vaccine derivative of FCV2.1, named FCV2.3.

Cloning of Truncated SARS-CoV-2 Spike Ectodomain into pRLF1 for Obtaining Non-Replicating Adenoviral Vaccine Vector Plasmid pFCV2.1

The truncated spike ectodomain gene was synthesized as eBlocks™ gene fragments (Integrated DNA Technologies) and then assembled together creating longer fragment containing CMV promoter and enhancer region, the truncated spike ectodomain, WPRE element, and a polyadenylation signal sequence by using Gibson assembly master mix (NEB, E2611) followed by PCR amplification of the assembled fragment. To assemble the newly made truncated spike ectodomain fragment into an adenoviral vector, the adenoviral vector genomic backbone plasmid pRLF1 was first linearized using BstZ17I restriction enzyme followed by ethanol precipitation. pRLF1 is an adenoviral vector genomic backbone plasmid developed by Rokote Laboratories Finland to suit Gibson assembly recombination of transgenes into the deleted E1 region.

To obtain pFCV2.1, the truncated spike ectodomain fragment was assembled into the digested viral backbone pRLF1 using Gibson assembly master mix (NEB, E2611) according to the manufacturer's instructions. The Gibson assembly reaction was then transformed into NEB® 5-alpha Competent E. coli (NEB, C2987H) according to manufacturer's instructions. Positive colonies were screened by PCR and the correct recombination events were further confirmed by sequencing the construct.

Final viral vaccine Drug Product will be stored in below −60° C. A long term stability study will be conducted to provide data for assigning the batch expiration date. Based on preliminary data available on adenoviruses stored in the proposed final formulation buffer, at least 3 years stability is expected.

### In Vitro Characterization of the Truncated Spike Ectodomain

### HEK293 Cell Line

Human embryonic kidney HEK293 cell line was cultured in DMEM supplemented with 10% FBS, 1% L-glutamine and 1% penicillin/streptomycin. Cells were cultivated at 37° C. in 5% CO₂ in a humidified atmosphere.

### HEK293 Transfections

2 μg of full-length adenoviral vaccine vector plasmid pFCV2.1 was used to transfect HEK293 cells using Trans-IT (MirusBio) transfection reagent according to manufacturer's instructions.

### Western Blot Analyses

Supernatant from pFCV2.1 transfected HEK293 cells were separated by sodium dodecyl sulfate-polyacrylamide gel electrophoresis and then blotted onto a nitrocellulose membrane (Bio-Rad). After being blocked 30 min at room temperature (RT) with a solution of phosphate-buffered saline and 0.05% Tween 20 containing 5% nonfat dry milk, the membranes were treated with COVID19 convalescent patient serum as a primary antibody and then with IRdye labelled secondary antibodies against human immunoglobulins (LI-COR Biosciences). The Odyssey infrared imaging system and Image Studio v3.1 software (LI-COR Biosciences) were utilized to visualize the blots.

### Virus Production

pFCV2.1 adenoviral vaccine vector plasmid was digested with PacI (New England Biolabs) to release the adenoviral genome. Digested viral genome was purified by ethanol precipitation and dissolved in TE buffer. 2 μg of the linearized viral genome was then transfected into HEK293 cells using Trans-IT (MirusBio) transfection reagent according to manufacturer's instructions.

Recovered virus from the transfection was then further amplified in HEK293 cells and purified using Adeno-X Maxi purification kit (Takara Bio) according to manufacturer's instructions.

### Animal Studies

### Housing Conditions and Animal Care

The temperature in the experimental animal room was between +19° C.-+25° C. The relative humidity was at least 30% and not exceed 70%. Lighting was artificial, 12 h light and 12 h dark in phase with the natural day/night cycle. Animals were housed individually (1 animal/cage). The animals were taken care of according to the Standard Operating Procedures (SOPs) of University of Turku Central Animal Laboratory. All animals were observed daily for general well-being, signs of morbidity and mortality. Particular attention was paid to clinical signs that may indicate any adverse effects on the cardiovascular, respiratory, or central nervous systems. Detailed clinical observations outside the home cage were done on dosing days as follows: once prior to dosing, right after dosing when the animal has woken up and 3-4 hours and approximately 24 hours after dosing. After dosing, detailed clinical observations were done once a week and on the day of the necropsy (study day 28).

### Study Design and Dose Groups

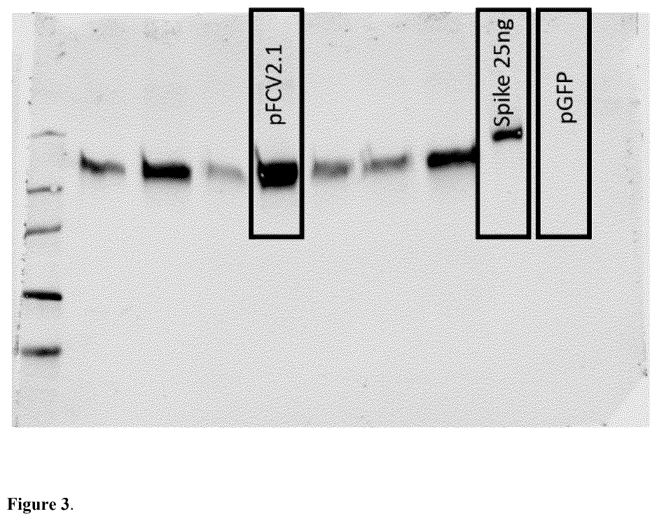

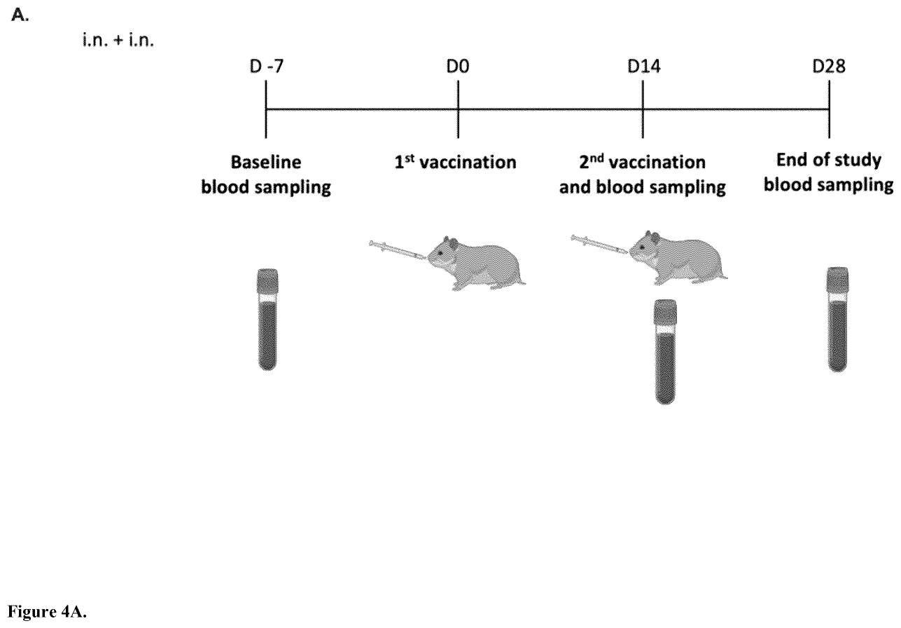

Immunogenicity of the product FCV2.1 was assessed using Syrian golden hamsters (Mesocricetus auratus) strain HsdHan®: AURA. FCV2.1 was administered intranasally in four different concentrations to two female and two male animals. Animals received the primary vaccination on study day 0 followed by booster dose on Study Day 14. The animals were weighed and assigned to groups based on random assignment, separately for males and females. At the start of the experiment animals were 34-36 weeks old and the weight variation of animals used in the study were less than +25% of the mean weight of each sex. Blood samples for IgG analysis were taken on study days-7 (baseline), 14 (two weeks after the primary dose) and 28 (two weeks after the booster dose). The results are shown in FIGS. 3 and 4.

FCV2.1 Efficacy Study in SARS-CoV-2 Challenged Syrian Golden Hamsters with Omicron BA.5 Variant

A total of 27 Golden Syrian Hamsters (approximately 50% male and 50% female) were divided into two groups as follows:
Group 1 (G1), the positive control group (n=12): non-vaccinated and challenged with a dose of 10⁴ TCID50 of the Omicron BA.5 variant of SARS-CoV-2.
Group 2 (G2), a group vaccinated with FCV2.1 (2 intranasal doses, 1×10¹⁰ viral particles per dose, 3 weeks apart, n=15) and challenged with a dose of 10⁴ TCID50 of the Omicron BA.5 variant of SARS-CoV-2 two weeks after the second immunization.

Clinical signs (including weight) of all animals were recorded daily from the day of SARS-CoV-2 challenge onwards. At 2-, 4-, and 7-days post-infection (dpi), 4 or 5 animals from each group were sacrificed at each time point. Necropsies were performed, and oropharyngeal (OP) swabs, blood, nasal turbinates, and lungs were collected, and viral RNA and infectious virus particles were analyzed. Immunohistochemistry analyses were conducted on nasal turbinates for SARS-CoV-2 nucleocapsid protein.

Vaccination Study with FCV2.3 in Balb/c Mice

Eight Balb/c mice were first vaccinated intramuscularly with Comirnaty (original, 2 ug/dose) and then divided in two groups of 4 animals. Group 1 received another intramuscular dose of Comirnaty (Original) 20 days after the first dose and Group 2 received intranasal booster with FCV2.3 20 days after the Comirnaty (Original priming dose). 3-weeks later blood was collected, and neutralizing antibodies were assessed against Wuhan, BA.1, BA.4/5 and XBB.

### CITATION LIST

### Patent Literature

WO2021045541A1
KR20210028116A

### Non Patent Literature

Chaudhary, J. K., Yadav, R., Chaudhary, P. K., Maurya, A., Kant, N., Rugaie, O. A., Haokip, H. R., Yadav, D., Roshan, R., Prasad, R., Chatrath, A., Singh, D., Jain, N., & Dhamija, P. (2021). Insights into COVID-19 Vaccine Development Based on Immunogenic Structural Proteins of SARS-CoV-2, Host Immune Responses, and Herd Immunity. Cells, 10 (11).

1. A viral vector comprising, at an insertion site in the viral genome, a nucleic acid sequence encoding a modified extracellular domain of the SARS-CoV-2 spike protein,
wherein said modified extracellular domain comprises an N-terminal signal peptide causing the spike protein to enter a secretory system in a host cell, and
wherein said modified extracellular domain comprises a C-terminal deletion of at least of heptad repeat 2 (HR2), transmembrane segment (TM) and cytoplasmic tail (CT) of the spike protein.

2. The viral vector according to claim 1, wherein the deletion site of said C-terminal deletion is located at the region corresponding to positions 1030-1157 of the omicron BA.5 strain spike protein of SEQ ID NO:1.

3. The viral vector according to claim 1, wherein said C-terminal deletion comprises part but not all of connector domain (CD) of the spike protein.

4. The viral vector according to claim 1, wherein said N-terminal signal peptide is the human interleukin-3 secretion signal peptide or the natural signal peptide of the SARS-CoV-2 spike protein.

5. The viral vector according to claim 1, wherein said modified extracellular domain of the SARS-CoV-2 spike protein comprises a mutated furin cleavage site with single amino acid modifications in positions R677G, R678S and R680S, wherein said positions corresponds to the respectively numbered amino acid positions of the omicron BA.5 strain spike protein of SEQ ID NO:1.

6. The viral vector according to claim 5, wherein said modified extracellular domain of the SARS-CoV-2 spike protein comprises the amino acid sequence of SEQ ID NO:2, amino acids 22-1142 of SEQ ID NO:2, or a sequence which has at least 80% sequence identity with the sequence of SEQ ID NO:2, or with amino acids 22-1142 of SEQ ID NO:2.

7. The viral vector according to claim 1, wherein said vector is selected from the group consisting of adenovirus vectors, adeno-associated virus vectors, retrovirusvectors, lentivirus vectors, herpex simplex virus vectors, poxvirus vectors, and orthomyxovirus vectors.

8. The viral vector according to claim 7, wherein said vector is an adenoviral vector.

9. The viral vector according to claim 1, wherein said modified extracellular domain of the SARS-CoV-2 spike protein comprises at least N-terminal domain (NTD), receptor-binding domain (RBD), fusion peptide (FP), heptad repeat 1 (HR1), and central helix (CH).

10. The viral vector according to claim 1, wherein said nucleic acid sequence encoding said modified extracellular domain is as set forth in SEQ ID NO: 3, or a nucleotide sequence having at least 70%, 80%, 85%, 90%, or 95% sequence identity with the nucleotide sequence as set forth in SEQ ID NO:3.

11. The viral vector according to claim 9, wherein said modified extracellular domain of the SARS-CoV-2 spike protein comprises in the C-terminal end one or more further receptor binding domains (RBD) from SARS-CoV-2 spike proteins which are immunologically distinct from said modified extracellular domain of the SARS-CoV-2 spike protein.

12. The viral vector according to claim 1, wherein said modified extracellular domain of the SARS-CoV-2 spike protein comprises in the C-terminal end a trimerization domain, human collagen trimerization domain, leuzine zipper domain or any other additional C-terminal amino acid sequence promoting trimerization of said spike protein.

13. The viral vector according to claim 1, wherein said modified extracellular domain of the SARS-CoV-2 spike protein comprises in the C-terminal end a transmembrane domain, or any other additional C-terminal amino acid sequence promoting transmembrane anchoring of said spike protein.

14. The viral vector according to claim 12, wherein said modified extracellular domain of the SARS-CoV-2 spike protein comprises in the C-terminal region in any order i) a trimerization domain, human collagen trimerization domain, leuzine zipper domain or any other additional C-terminal amino acid sequence promoting trimerization of said spike protein, and ii) a transmembrane domain, or any other additional C-terminal amino acid sequence promoting transmembrane anchoring of said spike protein.

15. The viral vector according to claim 11, wherein said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, and ii) a trimerization domain, human collagen trimerization domain, leuzine zipper domain or any other additional C-terminal amino acid sequence promoting trimerization of said spike protein.

16. The viral vector according to claim 11, wherein said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, and ii) a transmembrane domain, or any other additional C-terminal amino acid sequence promoting transmembrane anchoring of said spike protein.

17. The viral vector according to claim 11, wherein said modified extracellular domain of the SARS-CoV-2 spike protein comprises in the C-terminal end in any order i) one or more receptor binding domains (RBD) from SARS-CoV-2 spike proteins which are immunologically distinct from said modified extracellular domain of the SARS-CoV-2 spike protein, ii) a trimerization domain, human collagen trimerization domain, leuzine zipper domain or any other additional C-terminal amino acid sequence promoting trimerization of said spike protein, and iii) a transmembrane domain or any other additional C-terminal amino acid sequence promoting transmembrane anchoring of said spike protein.

18. A vaccine composition comprising the vector of claim 1 in a mucosal administration formulation.

19. The vaccine composition according to claim 18, wherein said mucosal administration formulation is selected from the group consisting of nasal drops, aerosols, sprays, powder sprays, gels, microspheres, liposomes, membranes, and suspensions.

20. The vaccine composition according to claim 19, wherein said vaccine composition is a spray or sprayable composition in a liquid dosage form.

21. The vaccine composition according to claim 18 further comprising a pharmaceutically-acceptable adjuvant, carrier, diluent or excipient.

22. (canceled)

23. (canceled)

24. The vaccine composition according to claim 18, wherein said composition is formulated for intranasal administration.

25. A method of preventing COVID-19 comprising administering intranasally, a prophylactically or therapeutically effective amount of the vaccine composition according to claim 18 to a mammal.

26.-31. (canceled)


SEQUENCE LISTING (3 sequences)

SEQ ID NO: 1 - protein, 1268 aa, Coronavirus SARS-CoV-2
MFVFLVLLPLVSSQCVNLITRTQSYTNSFTRGVYYPDKVFRSSVLHSTQDLFLPFFSNVTWFHAISGTNGIKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1142 aa, synthetic construct
MSRLPVLLLLQLLVRPGLQAPQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYD

SEQ ID NO: 3 - DNA, 3426 bp, synthetic construct
  source 1..3426: mol_type = other DNA
atgagccgcctgcccgtcctgctcctgctccaactcctggtccgccccggactccaagctccccagtgcgtcaacctgatcaccagaacccaaagctacaccaatagcttcacacggggggtgtactaccctgataaggtgttccggagttctgtgctgcactccacacaggacctgtttctgccttttttttccaatgtgacatggttccacgccatcagcggcaccaacggcacaaaaaggtttgacaaccccgtgctgccatttaatgatggggtgtacttcgcttccaccgagaaatccaacatcattaggggctggatcttcggcactaccctggattctaagactcagtccctgctgatcgtgaataacgccaccaacgtggtgatcaaggtgtgcgaatttcagttctgcaacgatccattcctggacgtgtactaccacaagaataacaagagctggatggagagcgagttccgcgtgtactcttccgcaaacaactgcacctttgaatatgtgagccagcccttcctgatggacctggagggaaagcagggcaatttcaaaaacctcagggaatttgtcttcaagaatatcgatggctatttcaagatctacagcaagcatactcccatcaacctgggcagagacctgccccagggcttcagcgctctggagcctctggtggacctgcccatcggcatcaacattaccagattccagaccctgctggctctgcatcggtcttacctgactcccggagatagcagcagcggctggaccgccggcgccgctgcctattatgtggggtacctgcagcctagaaccttcctgctgaagtacaacgagaacggcaccatcacagatgccgtggactgcgctctggacccactctccgagaccaaatgcaccctgaagtcctttaccgtggagaaaggcatctaccagacctccaatttcagagtgcagcctacagagagcatcgtgcgcttccccaacatcaccaacctgtgccctttcgacgaggtgttcaatgcaacacggtttgccagcgtgtacgcttggaatcggaagcggatcagcaactgcgtggccgactactctgtgctgtataacttcgccccctttttcgcctttaaatgttacggagtgtccccaactaagctgaacgacctgtgcttcacaaacgtgtatgctgatagcttcgtgattcggggcaacgaagtcagccagatcgctcccggacagactggaaacatcgccgactacaactacaagctgcctgacgacttcaccggctgcgtgatcgcctggaactccaacaagctggatagcaaggtgggcggcaattacaactaccggtatcgcctgttcagaaagtctaatctgaagccttttgagcgcgacatcagcaccgagatctatcaggccggaaacaagccctgcaacggagtggccggggtgaactgctactttcctctgcagagctatggctttcggcccacctacggggtggggcaccagccctaccgggtggtggtcctgtccttcgagctgctgcacgcccccgccaccgtgtgcggccccaagaaatccaccaacctggtgaagaataagtgtgtgaacttcaacttcaacggcctcaccggcaccggcgtgctgaccgagtctaataagaaattcctgcccttccagcagttcgggagggacatcgccgataccacagatgccgtcagagacccacagaccctggagatcctggacatcaccccctgctcattcggcggcgtgagcgtcatcacacctggcaccaacaccagcaatcaggttgccgtgctgtaccagggggtgaactgcaccgaggtgcctgtggccattcacgccgaccagctcacccctacttggcgggtgtacagcacaggctccaacgtgttccagaccagggccggatgcctgatcggcgccgagtacgtgaataattcctatgagtgcgacatccccatcggagccgggatttgcgccagctaccagacccagacaaagagccacggcagcgccagctccgtcgccagccagagcatcatcgcctacaccatgtccctgggggccgagaatagcgtcgcatacagtaataattccattgccatcccaaccaacttcaccatcagcgtgaccaccgaaattctgcccgtgtcaatgaccaaaacctccgtggactgtaccatgtacatctgtggcgacagcaccgagtgcagcaacctgctgctgcagtacggatccttttgcacccagctgaagcgggccctgacaggaatcgccgtggagcaggacaagaacacccaggaggtgttcgcccaggtgaaacagatctacaagaccccccccatcaagtacttcggtgggttcaacttcagccagatcctgccagacccctctaagccctccaagaggagctttatcgaagacctgctgttcaacaaggtcaccctggccgatgcaggcttcatcaagcagtacggggattgtctgggcgacatcgccgccagagacctgatctgtgcacagaagtttaacggcctgacagtcctgcctccactgctgactgacgagatgatcgcccagtatacctccgccctgctggccggaactattactagcggctggaccttcggcgccggtgccgctctgcagatcccctttgccatgcagatggcctatcgcttcaacggcattggagtgacccagaacgtgctgtatgagaaccagaagctgatcgccaaccagttcaactccgccatcggcaaaattcaggactctctgagctccactgccagtgccctgggcaagctgcaggatgtggtgaatcacaacgcccaggccctgaacacactcgtgaaacagctgtcctccaagttcggcgctattagcagcgtgctgaatgatatcctgagccggctggatccccccgaggccgaagtgcagattgatagactgattacaggcagactgcagagtctgcagacctacgtgactcagcagctgatccgcgctgccgagatcagagcctccgccaacctggccgccaccaagatgtccgagtgcgtgctgggccagtccaaaagagtggatttttgcggcaagggctaccacctgatgtcatttcctcagtctgctcctcacggcgtggtgttcctgcacgtgacctatgtgcccgcccaggagaagaactttaccactgcccctgccatctgccacgacggcaaggcccactttccccgcgagggcgtgttcgtgtctaacggaacccactggttcgtcacccagaggaatttttacgagccacagatcattaccacagataacacctttgtcagtggcaattgcgatgtggtgatcggcatcgtcaacaataccgtgtacgac